(19) United States
(12) Patent Application Publication
(10) Pub. No.: US 2009/0287277 A1
(43) Pub. Date: Nov. 19, 2009

(54) IMPLANTABLE NEUROSTIMULATION ELECTRODE INTERFACE

(75) Inventors: Brian M. Conn, Broomfield, CO (US); Denis Dupeyron, Mougins (FR)

Correspondence Address: MARSH, FISCHMANN & BREYFOGLE LLP, 8055 East Tufts Avenue, Suite 450, Denver, CO, 80237, US

(73) Assignee: Otologics, LLC, Boulder, CO (US)

(21) Appl. No.: 12/211,664

(22) Filed: Sep. 16, 2008

Related U.S. Application Data
(60) Provisional application No. 61/054,385, filed on May 19, 2008.

(51) Int. Cl. A61N 1/36; A61N 1/05

(52) U.S. Cl. 607/57; 607/137

(57) ABSTRACT

An implantable neurostimulation electrode interface and related system arrangements and methods are provided. The implantable interface may include at least a first stimulation signal channel for receiving a first electrode stimulation signal and a plurality of electrode signal channels electrically interconnected or interconnectable to a plurality of electrodes for neurostimulation. The interface may further include a router electrically interconnected to the first stimulation signal channel and to the plurality of electrode signal channels. The router is controllable to directly route the first electrode stimulation signal to different first successive sets of one or more of the plurality of electrode signal channels. The router may be adapted for routing the first electrode stimulation signal as an electrical current signal, without modification of the signal. Stimulation signal generation componentry and power source componentry may be located remotely from the implantable interface. Such componentry may be selectively interconnectable to and disconnectable from the implantable interface.

### RELATED APPLICATIONS

This application claims priority to U.S. Provisional Application Ser. No. 61/054,385, filed May 19, 2008, entitled “IMPLANTABLE NEUROSTIMULATION ELECTRODE INTERFACE”, the entirety of which is hereby incorporated by reference.

### FIELD OF THE INVENTION

The present invention relates to neurostimulation, and more particularly to an implantable electrode interface that facilitates the upgrade, servicing and replacement of interconnected system power and signal generation componentry. The invention is particularly apt for auditory neurostimulation applications, and simplifies a recipient's migration from a semi-implantable system to a fully implantable auditory neurostimulation system.

### BACKGROUND OF THE INVENTION

The utilization of neurostimulation implant devices is ever-increasing. Such devices utilize a plurality of implanted electrodes that are selectively activated to affect a desired neuro-response, including sound sensation, pain/tremor management, and urinary/anal incontinence. By way of primary interest, auditory neurostimulation implant devices include auditory brainstem implant (ABI) and cochlear implant (CI) devices.

In the case of CI devices an electrode array is inserted into the cochlea of a patient, e.g. typically into the scala tympani so as to access and follow the spiral currature of the cochlea. The array electrodes are selectively driven to stimulate the patient's auditory nerve endings to generate sound sensation. In this regard, a CI electrode array works by utilizing the tonotopic organization, or frequency-to-location mapping, of the basilar membrane of the inner ear. In a normal ear, sound vibrations in the air are transduced to physical vibrations of the basilar membrane inside the cochlea. High frequency sounds do not travel very far along the membrane, while lower frequency sounds pass further along. The movement of hair cells, located along the basilar membrane, creates an electrical disturbance, or potential, that can be picked up by auditory nerve endings that generate electrical action pulses that travel along the auditory nerve to the brainstem. In turn, the brain is able to interpret the nerve activity to determine which area of the basilar membrane is resonating, and therefore what sound frequency is being sensed. By directing which electrodes of a CI electrode array are activated, cochlear implants can selectively stimulate different parts of the cochlea and thereby convey different acoustic frequencies corresponding with a given audio input signal.

With ABI systems a plurality of electrodes may be implanted at a location that bypasses the cochlea. More particularly, an array of electrodes may be implanted at the cochlea nucleus, or auditory cortex, at the base of the brain to directly stimulate the brainstem of a patient. Again, the electrode array may be driven in relation to the tonotopic organization of a recipient's auditory cortex to obtain the desired sound sensation.

As may be appreciated, in the case of either ABI electrodes or CI electrodes, audio signals (e.g. from a microphone) may be processed, typically utilizing what is referred to as a speech processor, to generating stimulation signals utilized to selectively drive the electrodes for stimulated sound sensation. Further, in both implant approaches a source of power may be included to power the stimulation signal generator.

To date, implant modules utilized in CI and ABI systems have largely displayed architectures with dedicated functionality in relation to the inclusion of on-board speech processors and/or power sources. Such an approach has frustrated the ready implementation of improved power sources (e.g. batteries having improved storage capabilities) and/or upgrades for speech processors (e.g. speech processors having enhanced processing capabilities), as well recipient migration from semi-implantable systems to fully-implantable systems.

### SUMMARY OF THE INVENTION

In view of the foregoing, one objective of the present invention is to provide an implantable interface for neurostimulation electrodes that facilitates the replacement and/or upgrading of system power and/or stimulation signal generation componentry (e.g. stimulation processor).

Another objective of the present invention is to accommodate long-term implanted placement of a neurostimulator electrode array, and selective interconnection and disconnection thereof with other system componentry, wherein a recipient may initially utilize a semi-implantable system having one or more components located externally, and subsequently migrate to a fully-implantable system having all components implanted.

Yet another objective of the present invention is to reduce the size and complexity of implanted neurostimulation componentry, thereby simplifying surgical implant and servicing procedures, (e.g., neurostimulation componentry implanted within the auditory cortex of a recipient, at a spinal interface region of a patient, at a muscular interface of the urethra or anus region of a recipient, etc.).

One or more of the above objectives and additional advantages may be realized by an implantable electrode interface (e.g., an implanted hearing instrument interface) that comprises at least a first stimulation signal channel for receiving a first electrode stimulation signal (e.g. corresponding with an audio input signal) and a plurality of electrode signal channels electrically interconnected or interconnectable to a plurality of neurostimulation electrodes (e.g. an electrode array adapted for one of auditory brainstem and cochlear stimulation). The interface further includes a router electrically interconnected to the first stimulation signal channel and to the plurality of electrode signal channels, wherein the router is controllable to route the first electrode stimulation signal (e.g. on a dynamic basis) to different first successive sets of one or more of the plurality of electrode signal channels, wherein neurostimulation may be realized (e.g., auditory neuro stimulation).

The router may be adapted for receiving and directly routing the first electrode stimulation signal as an electrical current signal to the different first successive sets of one or more of the plurality electrode signal channels. In this regard, the router may be provided so that the stimulation current signal is routed without signal modification (e.g. without varying amplitude, pulse width, frequency or other current signal characteristics) and/or without signal buffering. Correspondingly, the implantable interface may avoid the inclusion of other stimulation signal generation componentry (e.g., speech processor) and power source componentry (e.g. a battery power source) that may be subject to replacement or upgrade servicing, and that otherwise consume space. In the later regard, the implantable interface may be of a relative small size, thereby facilitating semi-permanent positioning (e.g., on a cortical surface of a temporal bone of a recipient, within a mastoidectomy of a recipient or within a middle ear of a recipient adjacent to or near a cochlea of a recipient).

The noted stimulation signal generation and power source componentry may be remotely located from the implantable interface and operatively interconnected thereto. Further, direct current (DC) blocking componentry for blocking a DC portion of the first electrode stimulation signal may be located remotely from the implantable interface, thereby yielding further size reduction advantages at the implantable interface.

By way of example, in a fully implantable system arrangement the noted stimulation generation, power source and DC blocking componentry may be located in one or more implantable modules that are electrically interconnected to the implantable interface (e.g. via signal cable(s)). Such module(s) may be positioned at implant locations spaced, or remote, from the implantable interface where more space is available and where such modules are more accessible for replacement, servicing and upgrade procedures. For example, in auditory neurostimulation applications the module(s) may be positioned outside of the middle ear of a recipient (e.g. at a location on the cortical surface of the temporal bone near a mastoidectomy).

In a semi-implantable system arrangement for auditory neurostimulation, some or all of the noted componentry may be located externally, wherein an RF signal corresponding with an audio signal (e.g. an output signal from an externally located microphone) may be transcutaneously transmitted (e.g., between externally/internally located antennas (e.g., inductively coupled coils) and provided to the implantable interface. In such a system arrangement, a speech processor and/or a power source may be located either internally or externally (e.g. wherein a speech processor may process an audio signal to generate a stimulation signal that is transcutaneously transmitted as an RF signal).

The implantable interface may further include a biocompatible housing, wherein the router is sealably disposed within the housing. In turn, a first stimulation signal connector may be interconnected to the housing to define or at least partially define the first stimulation signal channel. The first stimulation signal connector may be adapted for selective interconnection of the router to and disconnection of the router from a first stimulation signal cable for carrying the first electrode stimulation signal. Such signal cable may be interconnected or interconnectable (e.g. via a plug-in connector) to an implanted speech processor for stimulation signal generation or to an implanted antenna (e.g., a coil) that is provided to transcutaneously receive an RF stimulation signal from an external antenna (e.g., a coil) that is interconnected to an externally-located stimulation signal generator (e.g., a speech processor).

The implantable interface may further include control logic, sealably disposed within the housing, for receiving a control signal and controlling the router in response thereto so as to route the first electrode stimulation signal to the different first successive sets of one or more of the plurality of electrode signal channels. By way of example, a digital processor and/or a plurality of gates may be employed for receiving a serial or multi-bit, digital control signal and for controlling the router in response thereto. The control signal may be generated by a control signal generator (e.g., a processor) that is remotely located from the implantable interface and operatively interconnected thereto. As may be appreciated, the stimulation signal generator and the control signal generator may be provided to an electrode to provide stimulation signals, and a control signal(s), respectively, in an operatively coordinated manner (e.g., in time correlation).

For example, in a fully-implantable system arrangement the control signal may be generated by a processor that is located in an implantable module that is electrically interconnected to the interface (e.g. via a signal cable). In one approach, such processor may be co-located with or otherwise defined by a speech processor utilized to generate the stimulation signal. Such module may be positioned at an implant location outside of the middle ear of a recipient. In a semi-implantable system arrangement the control signal may be generated by a processor that is externally located and transcutaneously transmitted as an RF signal via inductive coupling between externally/internally located coils and then directed to the implantable interface.

Relatedly, the implantable interface may include a control signal connector interconnected to the housing and adapted for selective connection of the processor to and disconnection of the processor from a control signal cable for carrying the control signal. Such signal cable may be interconnected or interconnectable (e.g. via a plug-in connector) to an implanted processor for control signal generation or to an implanted coil that is provided to transcutaneously receive an RF control signal from an external coil that is interconnected to an externally-located processor for control signal generation.

The implantable interface may further include a second stimulation signal channel for receiving a second electrode stimulation signal, wherein the second stimulation signal channel is electrically interconnected to the router. In turn, the router may be controllable to route the second electrode stimulation signal to different second successive sets of one or more of the plurality of electrode signal channels. In this regard, the second successive sets may be different than the first successive sets, wherein adjacent electrodes may be simultaneously driven by different stimulation signals to yield an intermediate sound frequency sensation (e.g. a sound sensation corresponding with a frequency that is between the frequencies corresponding with the driven, adjacent electrodes).

As may be appreciated, the implantable interface may include a plurality of stimulation signal channels for receiving a corresponding plurality electrode stimulation signals, wherein each of the plurality of stimulation signal channels is electrically interconnected to the router, and wherein the router is controllable to route each of the plurality stimulation signals to corresponding different successive sets of one or more of the plurality of electrode signal channels. In this regard, the router may be provided so that each of the plurality of stimulation signals may be routed as current signals free from modification of the signal characteristics thereof.

The implantable interface may further include a simple power circuit interconnected or interconnectable to a remotely located power source for receiving a power signal to power the router and/or the logic control noted hereinabove. In this regard, the power circuit may rectify an AC signal to DC power to supply the router, some additional componentry and optionally the stimulation current. The present invention further presents an inventive system for auditory neurostimulation. Such system comprises a stimulation signal generator for generating at least a first stimulation signal in response to an auditory signal; and an implantable interface having a router housed separately from said signal generator and electrically interconnected thereto, wherein the router is controllable to directly route said at least a first stimulation signal to different first successive sets of one or more of a plurality of electrode signal channels that are electrically interconnected or interconnectable to a plurality of electrodes for auditory neurostimulation.

In one aspect, the signal generator may be disposed in a bio-compatible, implantable module and the router may be disposed in a bio-compatible, implantable housing, wherein the implantable module and implantable housing may be electrically interconnected or interconnectable. By way of example, a cable line may operatively interconnect the signal generator disposed within the implantable module to the router disposed within the implantable housing. In this regard, the implantable housing may include a connector adapted for selective interconnection to and disconnection from a connector disposed on an end of the signal cable line that is interconnected to the implantable module. As may be appreciated, such an arrangement facilitates semi-permanent implant positioning of the implantable interface, while accommodating the removal and/or separate servicing of the speech processor disposed in the implantable module.

In a related aspect, the system may further include a power source disposed in a bio-compatible, implantable module, wherein the router may be disposed in a separate bio-compatible, implantable housing, and wherein the implantable module and implantable housing may be electrically interconnected or interconnectable. By way of example, a cable line may operatively interconnect the power source disposed within the implantable module to the router disposed within the implantable housing. In this regard, the implantable housing may include a connector adapted for selective interconnection to and disconnection from a connector disposed on the end of a signal cable line that is interconnected to the implantable module. In one embodiment the power source and stimulation signal generator may be disposed in the same implantable housing.

In an additional aspect, the system may further include a bio-compatible, implantable microphone that is interconnected or interconnectable to an implantable module having the signal generator disposed therewithin. The microphone may receive acoustic signals and output audio signals in response thereto for use by the signal generator in providing the electrode stimulation signal.

In yet a further aspect, the system may comprise an implantable antenna (e.g., coil) and an externally-locatable antenna (e.g., coil) that are adapted for wireless signal transmission) therebetween (e.g., via inductive coupling), wherein signals may be transcutaneously transmitted between the antennas. In conjunction with such an arrangement, the signal generator and a microphone may be located externally, wherein an audio signal output from the microphone may be utilized by the signal generator (e.g. a speech processor) to generate the stimulation signal. In turn, the external antenna may transmit the stimulation signal to the implanted antenna. The implantable interface may be interconnected to the implantable antenna for receipt of the stimulation signal. In conjunction with this approach, a power source may be externally located and interconnected to the microphone and signal generator.

In conjunction with the various system embodiments described above, the implantable interface may comprise one or more of the implanted interface features also described above. Further, the system may include a plurality of auditory neurostimulation electrodes electrically interconnected to the implantable interface in a manner that accommodates implanted positioning of the electrodes contemporaneous with positioning of the implantable interface. By way of example, the implantable interface may be integrated with a connector that is interconnected to one end of an electrode array. In turn, the integrated connector may be readily interconnected to and disconnected from a signal cable line(s) that is interconnected to an implantable simulation signal generator and power source.

As may be appreciated, the present invention further comprises an inventive method for driving a plurality of electrodes for auditory neurostimulation. The method includes the step of receiving at least a first electrode stimulation signal at an implantable electrode interface, and routing the first electrode stimulation signal at the interface to different successive sets of one or more of a plurality of electrodes for neurostimulation.

The routing step may include the step of controlling a router to dynamically define the different first successive sets of the plurality of electrodes. In turn, such controlling step may include processing a digital control signal. In one approach, the routing and processing steps may be completed at the implantable hearing instrument interface. In conjunction with such approach, the receiving step may include selectively interconnecting a first stimulation signal cable to the router at the implantable interface. In conjunction with the noted approach, the method may further include generating the first electrode stimulation signal at a processor that is located separate from the implantable electrode interface.

In conjunction with the control of the router, the router may be adapted to route the first electric stimulation signal as an electrical current signal to the different successive sets of one or more of the plurality of electrodes free from modification of the first electrode stimulation signal. In this regard, the routing step may be completed by utilizing the router to direct the first electrode stimulation signal to the different first successive sets of the plurality of electrodes.

In conjunction with the inventive method a second electrode stimulation signal may be received at the implantable interface and routed at the interface to different second successive sets of one or more of the plurality of electrodes. In this regard, the first electrode stimulation signal and second electrode stimulation signal may be employed to activate, in overlapping timed-relation first and second neurostimulation electrodes. For example, for auditory neurostimulation, the first and second electrodes may be located to stimulate nerve endings associated with first and second acoustic frequency stimulation, respectively, wherein sound sensation at an intermediate frequency (e.g. between the first and second frequencies) may be realized.

In a further aspect of the inventive method, the first electrode stimulation signal may be employed to drive the different first successive sets of one or more of a plurality of electrodes to effect neurostimulation, wherein a bodily generated electrical signal (e.g. generated by nerve endings in the cochlea of a recipient) received at one or more of the plurality of electrodes in response to the neurostimulation to generate a response signal. In turn, the response signal may be passed back through the implantable interface (either through the router, or digitally after having been digitized) for processing at a processor located separate from the implantable hearing instrument interface. In one approach, the response signal may be returned to a stimulation signal generator, wherein the response signal may be processed to measure the magnitude of neuroresponse to the electrode stimulation signal. In this regard, such measurements may be utilized in conjunction with fitting procedures in assessing appropriate signal characteristics to be employed in conjunction with the generation of electrode stimulation signals (e.g. setting the magnitude of pulses of comprising such signal).

In conjunction with the present invention, a further method is provided for auditory neurostimulation for an implant recipient. The method includes the steps of generating at least the first electrode stimulation signal implanted in a recipient at a first location (e.g. outside of the middle ear of a recipient in cochlear implant applications), and routing the first electrode stimulation signal at an implanted interface located at a second implanted location of the recipient (e.g. within the middle ear in a cochlear implant application) to different first successive sets of one or more of a plurality of electrodes for neurostimulation. In the later regard, the method may comprise a further step of positioning the plurality of electrodes at a third location within the recipient (e.g. within a cochlea of a recipient) in a cochlear implant application.

In one aspect, the inventive method may include the further step of locating a stimulation signal generator at said first location, wherein said locating said positioning steps are completed separately. In conjunction with this aspect the routing step may be completed utilizing a router located at the second location, wherein the method further includes electrically interconnecting said router and said signal generator after said positioning step.

In another aspect of the inventive method, the generating step may include the step of processing an audio signal utilizing an implanted speech processor. In conjunction with this aspect, the method may provide for receiving an acoustic signal at an implanted microphone to output an audio signal for use in the generating step. Further, the method may include the step of providing a power signal to a router to complete said routing step from an implanted power source.

In yet another further aspect, the generating step of the inventive method may include processing an audio signal at a stimulation signal generator externally located relative to a recipient. In this regard, the method may further include the step of receiving an acoustic signal at an externally located microphone or other audio source to generate the audio signal.

Additional aspects and advantages of the present will become apparent to those skilled in the art upon consideration of the further description that follows.

### BRIEF DESCRIPTION OF THE DRAWINGS

FIG. 1 is a schematic representation of one system embodiment comprising the present invention.

FIG. 2 is a schematic illustration of one semi-implantable system embodiment comprising the present invention.

FIG. 3A is a schematic illustration of another semi-implantable system and embodiment comprising the present invention.

FIG. 3B is a schematic illustration of the semi-implantable system embodiment of FIG. 3A, with an interface unit integrated into a connector of such embodiment.

FIG. 4 is a schematic illustration of one fully-implantable system embodiment comprising the present invention.

FIG. 4B is a schematic illustration of a fully-implantable system embodiment of FIG. 4A with an external accessory unit shown in conjunction therewith.

FIG. 4C is a schematic illustration of the fully implanted system embodiment of

FIGS. 4A and 4B, with an interface unit integrated into a connector thereof.

### DETAILED DESCRIPTION OF THE DRAWINGS

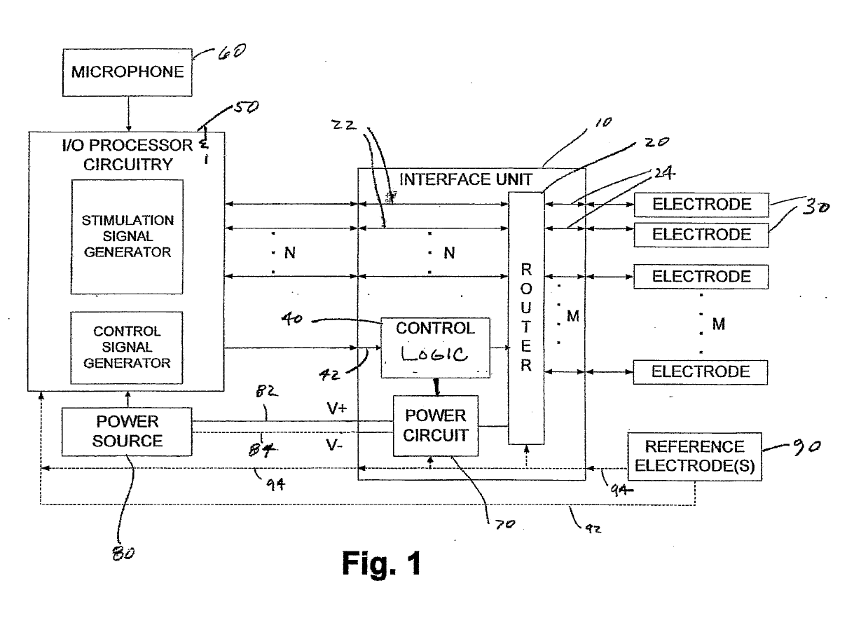

FIG. 1 illustrates one auditory neurostimulation system embodiment comprising the present invention. Other neurostimulation applications will be apparent to those skilled in the art.

As shown in FIG. 1, an implantable hearing instrument interface unit 10 may comprise a router 20 electrically interconnected with one to N stimulation signal channels 22 and electrically interconnected with one to M electrode signal channels 24. The router 20 may be selectively controllable to route one or more stimulation signals received from one or more of the stimulation signal channels 22 to one or more of the electrode signal channels 24. In turn, each routed stimulation signal may be employed to drive one or more electrodes 30 for neurostimulation. In this regard, the router 20 may be provided so as to route one or more electrode stimulation signal(s) as current signals without changing the amplitude, frequency or width of pulses comprising the current signal, and without otherwise buffering the current signal(s).

For purposes of controlling the router 20, the implantable hearing instrument interface 10 may comprise control logic 40 that is electrically interconnected to at least one control signal channel 42 for receiving a control signal. The control logic 40 may be electrically interconnected to router 20 so as to use the control signal to control the routing of one or more electrode stimulation signals received via one or more stimulation signal channels 22 to one or more electrodes 30 via one or more electrode signal channels 24. In this regard, the control signal may comprise a digital signal and the control logic 40 may be a digital logic. That is, the digital logic may comprise gates that interpret serial or parallel data provided on the control signal channel 42 in order to control the stimulation to be provided, for example which electrode is selected for the current stimulation.

As shown in FIG. 1, the stimulation signal channel(s) 22 and control signal channel 42 may be electrically interconnected to an input/output (I/O) processor and circuitry 50. The I/O processor and circuitry 50 may include a stimulation signal generator 52 (e.g. a speech processor) for generating the electrode stimulation signal(s) received at router 10 and a control signal generator 54 for generating the control signal received at control logic 40. Operation of the stimulation signal generator 52 and control signal generator 54 may be responsive to audio input signals received at the I/O processor and circuitry 50, as generated by a microphone 60.

The I/O processor and circuitry 50 may further comprise DC blocking componentry. For example, capacitors may be employed to prevent DC current from flowing while allowing AC current to flow. Alternatively, schemes using coulomb counters may be employed to ensure balanced current flow with no net DC current realized.

As further shown in FIG. 1, the implantable hearing instrument interface unit 10 may further comprise a power circuit 70 interconnected to control signal processor 40 and router 20 for directing power thereto. In turn, a separate power source 80 may be provided for providing a power signal to the power circuit 70. For example, power circuit 70 may be interconnected to a positive voltage supply line 82 and optionally connected to a negative voltage supply line 84 that are interconnected to the power source 80. The power circuit 70 may comprise diodes and capacitors to rectify an AC signal to DC power, or any other type of AC to DC and/or DC to DC converter.

In one implementation, two stimulation signal channels 22, one control signal channel 42 and positive/negative voltage supply lines 82, 84 may be provided. In turn, five electrical conductor lines may be provided via multiple cables or a single cable having five lines and a five-pin connector for selective interconnection to a five pin connector at the interface unit 10.

In the embodiment illustrated in FIG. 1, one or more reference electrode(s) 90 may be electrically interconnected to the I/O processor 50. Such interconnection may be realized in a number of alternate ways. As illustrated, the reference electrode(s) 90 may be interconnected to the I/O processor and circuitry 50 via one or more connection lines 92 that extend between the reference electrode(s) 90 and the I/O processor 50. Alternatively, the reference electrode(s) 90 may be interconnected though the interface 10 to the I/O processor and circuitry 50 via connection lines 94. In yet a further option, the reference electrode(s) 90 may be interconnected thought the I/O processor 50 via the router 20 of the interface 10 wherein one or more of the stimulation signal channels 22 may be utilized. As another option, the reference electrode(s) 90 may be interconnected to power circuit 70 that may be interconnected to power source 80 that may be interconnected to I/O processor and circuit 50.

In this regard, the embodiment shown in FIG. 1 may be provided and controlled to provide for monopolar stimulation, common ground stimulation or bipolar stimulation. For example, one electrode (e₁) from the set of M electrodes may be selected under the control of the control signal logic. Current may be provided to electrode e₁ with a current return path through an electrical reference electrode. This mode of stimulation is called “monopolar.” Alternatively, if one electrode (e₂) from the set of M electrodes is selected to provide stimulation current and the remaining electrodes in the set of M electrodes are electrically connected to the electrical reference, then this mode of stimulation is called common ground. Finally, if two electrodes (e₁ and e₂) from the set of M electrodes are selected to provide stimulation such that in an alternating manner the first electrode e₁ is electrically connected to the stimulation current source and e₂ is electrically connected to the electrical reference and subsequently e₂ is electrically connected to the stimulation current source and e₁ is electrically connected to the electrical reference, then this stimulation mode is bipolar stimulation. In all of these stimulation schemes, balanced anodic and cathodic stimulation should be provided.

Further, depending upon the number of stimulation signal channels 22 utilized, the embodiment may provide for simultaneous stimulation or pulsatile (e.g. non-simultaneous) stimulation. For example, under the control of the control signal logic, two electrodes may be selected to provide stimulation current such that unequal amounts of stimulation current are provided by the two electrodes (e.g., the current magnitudes are different). This bias in stimulation current will create an intermediate pitch perception for the patient between the two electrodes. The tonotopic location of the pitch perception can be controlled by the bias in the current between the two electrodes.

Additionally, the embodiment of FIG. 1 may be utilized during fitting procedures to measure bodily response to selectively activated electrodes 30. That is, under control of the control signal logic 40 the stimulation channels 22 may be routed to specific electrodes. A stimulas current is presented on the electrode by the stimulation signal generator in 50 and the response of the nerve to the stimulation signal is measured on the electrode by circuitry in the interface unit 10 or the I/O process or circuitry 50.

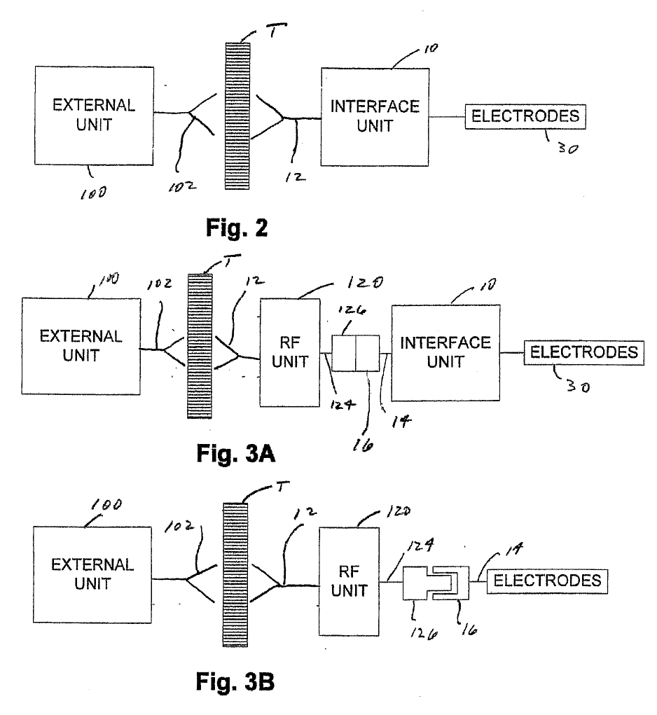

Reference is now made to FIGS. 2, 3A and 3B, and FIGS. 4A, 4B and 4C which illustrate various systems of embodiments of the present invention. As may be appreciated, such embodiments demonstrate a wide array of applications for implementation of the present invention.

In particular, FIG. 2 is directed to semi-implantable implementation in which an interface unit 10 is implanted together with interconnected or interconnectable electrodes 30 for neurostimulation in response to the operation of an operatively interconnected external unit 100. For such purposes, the interface unit 10 may comprise or otherwise be interconnected to an implanted antenna 12 (e.g., a coil) that is inductively coupleable to an external antenna 102 (e.g., a coil) comprising or otherwise interconnected to the external unit 100, wherein wireless radio frequency (RF) signals may be transcutaneously conveyed through tissue T. Such RF signals may be converted to electrical signals by antenna 12, wherein electrical signals from antenna 12 may be utilized at interface unit 10 to yield one or more electrode stimulation signals for stimulation of electrodes 30 and a power signal for powering the interface unit 10. In this regard, the external unit 100 may comprise a speech processor and circuitry for processing an audio signal (e.g. from a microphone interconnected to or integrated into the external unit 100) and for providing an output signal comprising a carrier component and a stimulation component. In turn, such output signal may be transcutaneously conveyed by external coil 102 to implanted antenna 12, wherein the carrier component may be utilized at interface unit 10 to power the interface unit 10 and the stimulation component may be routed as current signal at interface unit 10 to one or more of the electrodes 30 for neurostimulation. More particularly, the interface unit 10 may contain an RF-front end circuit which contains a rectification circuit to provide DC power to the power circuit in the interface unit 10. The RF-front end also contains circuitry to decode stimulation commands generated by the external unit. The commands are then routed from the stimulation lines 22 through the router 20 to the electrodes 30 under control of the control signal generator. As may be appreciated, the arrangement of FIG. 2 accommodates the readily implementation of speech processor upgrades in external unit 100, while also minimizing the number of implanted components.

Reference is now made to FIG. 3A which illustrates another semi-implantable implementation which is similar to the implementation of FIG. 2, with the difference being that the implanted circuitry provided for obtaining the power signal and electrode simulation signal from the transcutaneous RF signal may be located in an RF unit 120 that is separate from the interface unit 10. As shown, an implanted antenna 12 (e.g., a coil) may be interconnected to or otherwise provided as a part of the RF unit 120, wherein such componentry may be implanted at a first location (e.g., cortical surface of the temporal bone) and selectively interconnected and disconnected with an interface unit 10 that is implanted at a second location (e.g. near the mastoidectomy in the temporal bone). For example, a cable line 124 with connector 126 may be provided with RF unit 120 for selective connection to and disconnection from a cable line 14 with connector 16, provided with the interface unit 10. Such an arrangement facilitates permanent or semi-permanent positioning of interface unit 10 and electrodes 30, as well as the replacement of RF unit 120 with other componentry associated with a fully implantable system, as will be further described.

FIG. 3A illustrates an arrangement in which the connectors 16 and 126 are provided in separate housings from the interface unit 10 and RF unit 120. In another approach, by virtue of the limited functionality provided by interface unit 10, the interface unit 10 may be packaged with a connector as a single, integrated unit for selective interconnection with the connector 126 that is interconnected via cable 124 to the RF unit 120, as schematically shown in FIG. 3B. As may be appreciated, such capability further reduces size and implantation requirements associated with implanted componentry.

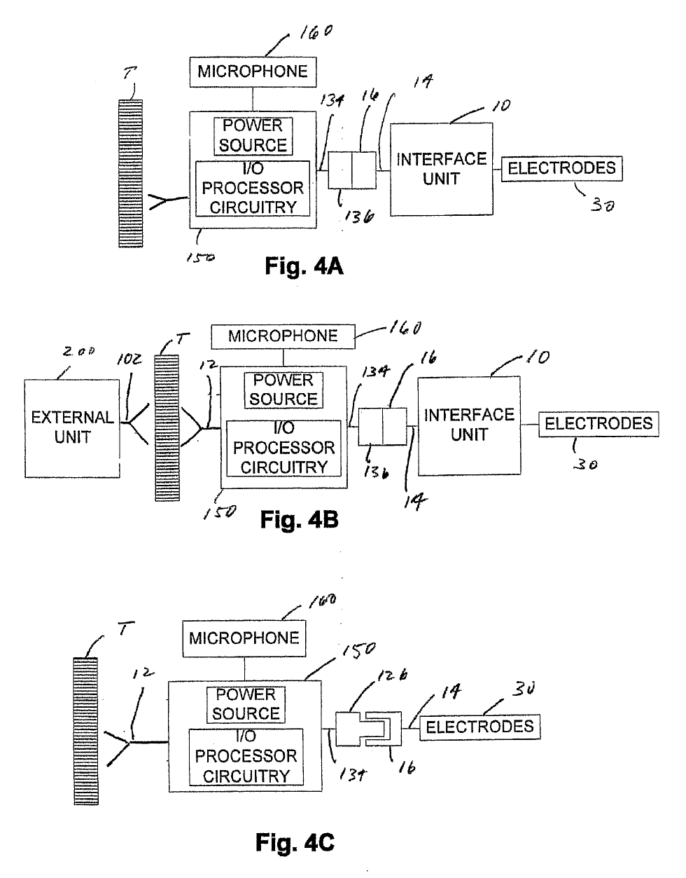

Reference is now made to FIG. 4A which illustrates a fully implantable system implementation of the present invention. In such an arrangement, the various componentry that may be included in or interconnected to the external unit 100 described above in relation to FIGS. 2, 3A and 3B may be implanted and selectively interconnected to and disconnected from an interface unit 10 and interconnected electrodes 30. In particular, power source and input/output (I/O) processor and associated circuitry, collectively 150, may be implanted in a common capsule or in two capsules. In turn, one or more of such capsules may be operatively interconnected via a cable 134 and connector 136 to a cable 14 and connector 16 that is interconnected to the interface unit 10. As illustrated, an implanted microphone 160 may further be interconnected to the one or more capsules for providing an audio signal to the I/O processor and circuitry 150 for use in the generation of the electrode stimulation signal(s). As shown, the module housing I/O processor and circuitry 50 may be interconnected or otherwise comprise a coil for use in receiving a transcutaneous signal from an antenna 112 interconnected to or otherwise provided with an external unit 200, as shown in FIG. 4B. In this arrangement, the external unit 200 may comprise componentry for recharging the implanted power source. Further, the external unit may be provided to convey software upgrades for a speech processor comprising the implanted I/O processor, and to provide other diagnostic functions. In this regard, external unit 200 may send data and commands to the I/O processor 150 and receive data back. The data may comprise diagnostic data describing the performance of the implant. The diagnostic data may also include the measurement of physiological parameters such as the impedance of the current path between electrodes of the evoked neural or brain response to electrical or acoustic stimuli.

It is again noted that, by virtue of the limited functionality provided by interface s unit 10, the interface unit 10 may be packaged with the connector as a single, integrated unit for selective interconnection with the connector 126 that is interconnected via cable 124 to the unit 50 schematically shown in FIG. 4C. As may be appreciated, such capability further reduces the size and implementation requirements associated with an implanted componentry.

What is claimed is:

1. An implantable electrode interface comprising:
a first stimulation signal channel for receiving a first electrode stimulation signal;
a plurality of electrode signal channels electrically interconnected or interconnectable to a plurality of electrodes for neurostimulation; and,
a router electrically interconnected to said first stimulation signal channel and to said plurality of electrode signal channels, wherein said router is controllable to directly route said first electrode stimulation signal to different first successive sets of one or more of said plurality of electrode signal channels.

2. An implantable electrode interface as recited in claim 1, further comprising:
a biocompatible housing, wherein said router is sealably disposed within said housing.

3. An implantable electrode interface as recited in claim 2, further comprising:
a first stimulation signal connector interconnected to said housing and defining said first stimulation signal channel, wherein said first stimulation signal connector is adapted for selective interconnection of said router to and disconnection of said router from a first stimulation signal cable for carrying said first electrode stimulation signal.

4. An implantable electrode interface as recited in claim 2, further comprising:
control logic, sealably disposed within said housing, for receiving a control signal and controlling said router in response thereto so as to route said first electrode stimulation signal to said different successive sets of one or more of said plurality of electrode signal channels.

5. An implantable electrode interface as recited in claim 4, further comprising:
a control signal connector interconnected to said housing, wherein said control signal connector is adapted for selective connection of said control logic to and disconnection of said control logic from a control signal cable for carrying said control signal.

6. An implantable electrode interface as recited in claim 1, wherein said router is adapted for routing said first electrode stimulation signal as an electrical current signal to said different successive sets of one or more of said plurality of electrode signal channels free from modification of said first electrode stimulation signal.

7. An implantable electrode interface as recited in claim 1, further comprising:
a second stimulation signal channel for receiving a second electrode stimulation signal, said second stimulation signal channel being electrically interconnected to said router, wherein said router is controllable to route said second electrode stimulation signal to different second successive sets of one or more of said plurality of electrode signal channels.

8. An implantable electrode interface as recited in claim 1, further comprising:
said plurality of electrodes arranged in an array and mounted to a carrier adapted for implanted positioning.

9. An implantable electrode interface as recited in claim 8, wherein said plurality of electrode signal channels comprise electrical signal wires mounted to said carrier, wherein each of said plurality of electrical signal wires are interconnected to different sets of one or more of said plurality of electrodes.

10. A method for driving a plurality of electrodes for neurostimulation, comprising:
receiving at least a first electrode stimulation signal at an implantable electrode interface; and,
routing said first electrode stimulation signal at said interface to different first successive sets of one or more of a plurality of electrodes for neurostimulation.

11. A method as recited in claim 10, wherein said routing step comprises:
controlling a router to dynamically define said different first successive sets of said plurality of electrodes.

12. A method as recited in claim 11, wherein said controlling step comprises:
utilizing a digital control signal.

13. A method as recited in claim 12, further comprising:
completing said routing and processing steps at said implantable electrode interface.

14. A method as recited in claim 13, wherein said receiving step comprises:
selectively interconnecting a first stimulation signal cable to said router at said implantable electrode interface.

15. A method as recited in claim 13, further comprising:
generating said first electrode stimulation signal at a processor located separate from said implantable hearing instrument interface.

16. A method as recited in claim 11, wherein said router is adapted for routing said first electrode stimulation signal as an electrical current signal to said different first successive sets of one or more of said plurality of electrodes free from modification of said first electrode stimulation signal.

17. A method as recited in claim 10, further comprising:
receiving a second electrode stimulation signal at said implantable electrode interface; and,
routing said second electrode stimulation signal at said interface to different second successive sets of one or more of said plurality of electrodes for neurostimulation.

18. A method as recited in claim 10, further comprising:
driving said different first successive sets of one or more of a plurality of electrodes with the said first electrode stimulation signal for neurostimulation.

19. A method as recited in claim 18, further comprising:
receiving a bodily-generated electrical signal at one or more of said plurality of electrodes in response to said neurostimulation to generate a response signal; and,
passing said response signal through said implantable interface for processing at a processor located separate from said implantable interface.
